(19) United States
(12) Patent Application Publication
(10) Pub. No.: US 2017/0003292 A1
(43) Pub. Date: Jan. 5, 2017

(54) PAXIP1 AS A BIOMARKER FOR WEE1 INHIBITOR THERAPY

(71) Applicant: H. LEE MOFFITT CANCER CENTER AND RESEARCH INSTITUT INC., Tampa, FL (US)

(72) Inventors: Alvaro N. Monteiro, Tampa, FL (US); Uwe Rix, Tampa, FL (US); Ankita Jhuraney, Kirkland, WA (US)

(21) Appl. No.: 15/125,629

(22) PCT Filed: Mar. 13, 2015
(86) PCT No.: PCT/US15/20339

§ 371 Date: Sep. 13, 2016

Related U.S. Application Data
(60) Provisional application No. 61/952,757, filed on Mar. 13, 2014.

(51) Int. Cl. G01N 33/574

(52) U.S. Cl. CPC G01N 33/57492; G01N 2800/52

(57) ABSTRACT

Disclosed is a method for selecting a cancer therapeutic for a patient that involves assaying a tumor biopsy sample from the subject to detect PAXIP1 expression, and selecting a WEE1 inhibitor as the cancer therapeutic if PAXIP1 is detected in the tumor biopsy sample.

### CROSS-REFERENCE TO RELATED APPLICATIONS

This application claims benefit of U.S. Provisional Application No. 61/952,757, filed Mar. 13, 2014, which is hereby incorporated herein by reference in its entirety.

### BACKGROUND

Many of the most prevalent for of human cancer resist effective chemotherapeutic intervention. Some tumor populations appear to have drug-resistant cells at the outset of treatment. In other cases, resistance appears to be acquired in much the same way as microbial resistance. Whatever the cause, resistance often terminates the usefulness of an antineoplastic drug. Chemosensitizers are urgently needed to improve the outcome of chemotherapy. Moreover, biomarkers are needed to predict the efficacy of chemotherapeutics for personalized treatment.

### SUMMARY

WEE1 inhibition can sensitize some, but not all, tumors to chemotherapy. Unfortunately, WEE1 levels by themselves do not predict this response. Disclosed herein is a biomarker (PAXIP1) that can be used to identify tumors that exhibit a favorable response to WEE1 inhibition. Therefore, a method is disclosed for selecting a cancer therapeutic for a patient, that involves assaying a tumor biopsy sample from the subject to detect PAXIP1 expression, and selecting a WEE1 inhibitor as a cancer therapeutic if PAXIP1 is detectable in the biopsy sample. In some embodiments, the method can further involve assaying the sample from the subject to detect WEE1 expression, and selecting a WEE1 inhibitor as the cancer therapeutic if PAXIP1 and WEE1 are present in the biopsy sample. In some embodiments, a WEE1 inhibitor is selected if the levels of PAXIP1 and/or WEE1 are elevated compared to a control level.

In some embodiments, the presence of PAXIP1 is an indication that WEE1 will effectively chemosensitize the subject's tumor to an antineoplastic agent. Therefore, in some embodiments, the method further involves treating the subject with therapeutically effective amounts of a WEE1-sensitive antineoplastic agent along with the WEE1 inhibitor.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a blot showing that endogenous WEE1 interacts with C-terminal tBRCT of PAXIP1.

FIG. 2 is a series of blots showing PAXIP1, WEE1, and p-CDC2 (a.k.a. p-CDK1) levels in lung cancer cell lines treated with AZD1775 and Cisplatin. D: DMSO; M: AZD1775 only; C: Cisplatin only; M+C: AZD1775 and Cisplatin combination.

FIG. 3 is a bar graph and table showing that overexpression of full length PAXIP1 leads to increased mitotic index at the G2/M checkpoint after AZD1775 treatment.

FIG. 4 is a bar graph showing that overexpression of full length PAXIP1 leads to sustained increase in apoptosis upon treatment with AZD1775 when compared to either the treatment alone or without PAXIP1 overexpression.

FIGS. 5A-5H show that cells that express both PAXIP1 and WEE1 exhibit synergy with AZD1775 and cisplatin treatment. Lung adenocarcinoma cell lines 1-H322 (FIGS. 5A, 5B), H1648 (FIGS. 5E, 5F) and H1395 (FIGS. 5G, 5H) and a squamous lung cancer cell line H157 (FIG. 5C, 5D) were treated with AZD1775 and cisplatin either alone or in combination for 1 h and p-CDK1, PAXIP1 and WEE1 levels were measured (FIGS. 5A, 5C, 5E, 5H). Cell lines were treated with AZD1775 and cisplatin for 72 h, cell viability was measured by CellTiterGlo and synergy scores were calculated (FIGS. 5B, 5D, 5F, 5H). Depicted in FIGS. 5B, 5D, 5F, and 5H are the three-dimensional dose-response surface curves with various combinations of drug concentrations (left) and the deviation from expected additive values determined by Bliss model of independence (right).

FIG. 6 illustrates the prevalence of PAXIP1 and WEE1 staining in tissue microarrays containing 106 lung tumors from patients stained using IHC for PAXIP1 and WEE1. The prevalence of PAXIP1 and WEE1 positive tumors is 31%.

FIGS. 7A-7D show WEE1 and PAXIP1 positive patient-derived xenografts (PDX) exhibit synergy with AZD1775 and cisplatin. Two tumors that were selected from a TMA of 68 lung tumors for 3-D clonogenic assays to test synergy of the combination of AZD1775 and cisplatin in these tumors. FIGS. 7A and 7C show percentage of tumor inhibition with different concentrations of AZD1775 and cisplatin. FIGS. 7B and 7D show combination index (CI) values obtained by applying the chou-talalay analysis to the percent inhibition values.

### DETAILED DESCRIPTION

Disclosed is a method for selecting a cancer therapeutic for a patient, comprising assaying a sample from the subject to detect PAXIP1, and selecting a WEE1 inhibitor as the cancer therapeutic if PAXIP1 is detected in the tissue sample. In some embodiments, the method can further involve assaying the sample from the subject to detect WEE1 expression, and selecting a WEE1 inhibitor as the cancer therapeutic if PAXIP1 and WEE1 are present in the biopsy sample. In some embodiments, a WEE1 inhibitor is selected if the levels of PAXIP1 and/or WEE1 are elevated compared to a control level.

For example, the WEE1 inhibitor can be AZD1775 (formerly MK-1775) (CAS# 955365-80-7) (2-allyl-1-(6-(2-hydroxypropan-2-yl)pyridin-2-yl)-6-((4-(4-methylpiperazin-1-yl)phenyl)amino)-1H-pyrazolo[3,4-d]pyrimidin-3(2H)-one), which has the following structure:

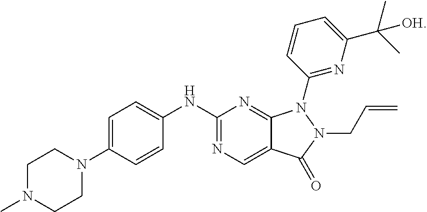 [SMILES: C=CCn1c(=O)c2cnc(Nc3ccc(N4CCN(C)CC4)cc3)nc2n1-c1cccc(C(C)(C)O)n1]

AZD1775 has been described in U.S. Pat. No. 7,834,019, and in PCT international Publications WO 2007/126122, WO 2007/126128 and WO 2008/153207, which are incorporated by reference herein in their entirety. Crystalline forms of MK-1775 are described in US Publication US2010/0124544 and PCT International Publication WO 2011/034743, which are incorporated by reference herein in their entirety.

In some embodiments, the WEE1 inhibitor is MK-3652, the structure of which is as shown below.

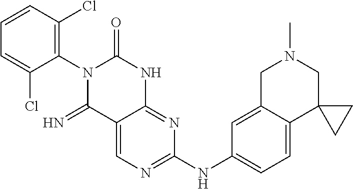 [SMILES: CN1Cc2cc(Nc3ncc4c(=N)n(-c5c(Cl)cccc5Cl)c(=O)[nH]c4n3)ccc2C2(CC2)C1]

MK-3652 is a WEE1 inhibitor which is useful for the treatment of cancer. MK-3652 has been described in PCT International Publication WO 2008/153207 and US Publication US2011/0135601, which are incorporated by reference herein in their entirety. Crystalline forms of MK-3652 are described in International Publication WO2009/151997 and US Publication US2011/0092520, which are incorporated by reference herein in their entirety.

The cancer of the disclosed methods can be any cell in a subject undergoing unregulated growth, invasion, or metastasis. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. In preferred embodiments, the cancer is non-small lung cancer or ovarian cancer.

In some embodiments, the cancer of the disclosed methods is a p53-deficient tumor cell. Inhibition of WEE1 activity prevents the phosphorylation of CDC2 and impairs the G2 DNA damage checkpoint. This can lead to apoptosis upon treatment with DNA damaging chemotherapeutic agents. Unlike normal cells, most p53-deficient or mutated human cancers lack the G1 checkpoint as p53 is the key regulator of the G1 checkpoint and these cells rely on the G2 checkpoint for DNA repair to damaged cells. Annulment of the G2 checkpoint may therefore make p53-deficient tumor cells more vulnerable to antineoplastic agents and enhance their cytotoxic effect.

As WEE1 inhibitors can act as a chemosensitizing agent for certain chemotherapeutic agents, in some embodiments, the method involves selecting a WEE1 inhibitor and a WEE1-sensitive antineoplastic drug, such as a DNA damaging chemotherapeutic agent, as the cancer therapeutics if PAXIP1 is present in the tumor biopsy sample. For example, the method can further involve treating the subject with a DNA damaging chemotherapeutic agent along with the WEE1 inhibitor if PAXIP1 is detected in the tumor biopsy sample.

In cases where PAXIP1 is not detected in the tumor, the method can involve selecting an alternative remedial or palliative treatment. For example, the method can involve selecting an antineoplastic drug that is not WEE1-sensitive. The choice between a remedial and palliative treatment is often one of degree. For example, a chemotherapeutic anti-neoplastic drug may be either palliative or remedial, depending on the dosage administered. A palliative treatment or dosage would have as its primary purpose that of reducing pain and discomfort. A remedial treatment or dosage would have as its primary purpose the destruction of remaining cancer cells, i.e., to prolong life. In the latter, case, the drug or dosage may actually decrease quality of life during the treatment period.

Chemotherapy drugs can be divided into several groups based on factors such as how they work, their chemical structure, and their relationship to another drug. Because some drugs act in more than one way, they may belong to more than one group. Alkylating agents directly damage DNA to prevent the cancer cell from reproducing. Therefore, in some embodiments, the WEE1-sensitive or WEE1-insensitive chemotherapeutic agent is a DNA damaging chemotherapeutic agent. For example, in some embodiments, the DNA damaging chemotherapeutic agents is a platinum-based antineoplastic agent, such as carboplatin or cisplatin.

Antimetabolites are a class of drugs that interfere with DNA and RNA growth by substituting for the normal building blocks of RNA and DNA. Therefore, in some embodiments, the WEE1-sensitive or WEE1-insensitive chemotherapeutic agent is an antimetabolite chemotherapeutic agent, such as 5-fluorouracil (5-FU), 6-mercaptopurine (6-MP), Capecitabine (Xeloda®), Cladribine, Clofarabine, Cytarabine (Ara-C®), Roxuridine, Fludarabine, Gemcitabine (Gemzar®), Hydroxyurea, Methotrexate, Pemetrexed (Alimta®), Pentostatin, or Thioguanine.

Anthracyclines are anti-tumor antibiotics that interfere with enzymes involved in DNA replication. Therefore, in some embodiments, the WEE1-sensitive or WEE1-insensitive chemotherapeutic agent is an anthracycline, such as Daunorubicin, Doxorubicin (Adriamycint), Epirubicin, or Idarubicin. In some embodiments, the chemotherapeutic agent is a non-anthracycline anti-tumor antibiotics, such as Actinomycin-D, Bleomycin, or Mitomycin-C. In some embodiments, the chemotherapeutic agent is the anti-tumor antibiotic Mitoxantrone.

Topoisomerase inhibitors interfere with enzymes called topoisomerases, which help separate the strands of DNA an they can be copied. Therefore, in some embodiments, the WEE1-sensitive or WEE1-insensitive chemotherapeutic agent is a topoisomerase inhibitors, such as topotecan, or irinotecan (CPT-11), etoposide (VP-16), or teniposide.

Mitotic inhibitors are often plant alkaloids and other compounds derived from natural products. They can stop mitosis or inhibit enzymes from making proteins needed for cell reproduction. Therefore, in some embodiments, the WEE1-sensitive or WEE1 -insensitive chemotherapeutic agent is a mitotic inhibitors, such as pactitaxel (Taxol®), docetaxel (Taxotere®), ixabepilone (Ixempra®), vinblastine (Velban®), vincristine (Oncovin®), vinorelbine (Navelbine®), or Estramustine (Emcyt®).

In some aspects, the method involves the use of an immunoassay to detect levels of PAXIP1, WEE1, or combinations thereof. Many types and formats of immunoassays are known that can be used for detecting the disclosed biomarkers. Examples of immunoassays are immunohistochemistry (IHC), enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time tong enough for the antibodies to form immune complexes with, i.e., to bind to, any molecules e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes funned during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biatimistreptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

A modifier unit such as a radionuclide can be incorporated into or attached directly to any of the compounds described herein by halogenation. In another aspect, the radionuclide can be attached to a linking group or bound by a chelating group, which is then attached to the compound directly or by means of a linker. Radiolabeling techniques such as these are routinely used in the radiopharmaceutical industry.

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

In some aspects, the method comprises detecting PAXIP1 and/or WEE1 gene expression by detecting PAXIP1 and/or WEE1 mRNA, A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, Nanostring Technologies nCounter®, expression microarrays, or reverse transcription-polymerase chain reaction (RT-PCR).

In theory, each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and RT-PCR is the most sensitive method for detecting and quantitating gene expression.

Northern analysis presents several advantages over the other techniques. The most compelling of these is that it is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides, Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is an extremely sensitive method for the detection and quantitation of specific mRNAs. The basis of the NPA is solution hybridization of an antisense probe (radiolabeted or nonisotropic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific rnRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

RT-PCR has revolutionized the study of gene expression. It is now theoretically possible to detect the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR. As with NPAs, RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PER reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

### Definitions

The term “subject” refers to any individual who is the target of administration or treatment. The subject can be a vertebrate, for example, a mammal. Thus, the subject can be a human or veterinary patient. The term “patient” refers to a subject that is under the care of a treating clinician (e.g., physician).

The term “sample from a subject” refers to a tissue (e.g., tissue/tumor biopsy), organ, cell (including a cell maintained in culture), cell lysate (or lysate fraction), biomolecule derived from a cell or cellular material (e.g. a polypeptide or nucleic acid), or body fluid from a subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed fir the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “palliative” refers to a treatment that is designed to relieve symptoms (e.g., reduce pain or discomfort) without having a curative effect on the underlying disease or cause (e.g., cell growth and metastasis).

The term “remedial” refers to a treatment that is designed to have a curative effect on the underlying disease or cause (e.g., cell growth and metastasis) and not just to relieve symptoms.

The term “neoplastic cells” refers to a cell undergoing abnormal cell proliferation (“neoplasia”). The growth of neoplastic cells exceeds and is not coordinated with that of the normal tissues around it. The growth typically persists in the same excessive manner even after cessation of the stimuli, and typically causes formation of a tumor.

The term “tumor” or “neoplasm” refers to an abnormal mass of tissue containing neoplastic cells. Neoplasms and tumors may be benign, premalignant, or malignant.

The term “cancer” or “malignant neoplasm” refers to a cell that displays uncontrolled growth, invasion upon adjacent tissues, and often metastasis to other locations of the body.

The term “metastasis” refers to the spread of malignant tumor cells from one organ or part to another non-adjacent organ or part. Cancer cells can “break away,” “leak,” or “spill” from a primary tumor, enter lymphatic and blood vessels, circulate through the bloodstream, and settle down to grow within normal tissues elsewhere in the body. When tumor cells metastasize, the new tumor is called a secondary or metastatic cancer or tumor.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### PAXIP1 as a Potential Biomarker for WEE1 Inhibitor Therapy

Cells have developed an elaborate molecular network to detect and repair DNA lesions. The DNA damage response (DDR) ensures DNA integrity by utilizing complex signaling events to coordinate recruitment of scaffolding proteins and repair enzymes to resolve the damage. Many of the DDR scaffolding proteins contain BRCA1 C-terminal (BRCT) modular domains, which are directed to damaged DNA by binding phosphorylated epitopes modified by DDR-dependent kinases such as ATM AIR, and DNA-PK. These domains predominantly occur as singleton or tandem domains (tBRCT). tBRCT domains have been used to perform a systematic analysis of protein-protein interactions (PPIN) in the DDR network.

Since posttranslational phosphorylations are used by the DDR to coordinate cellular response to damage, 18 kinases found in this tBRCT PPIN were examined using a small-molecule inhibitor screen. This screen that identified the inhibition of WEE1 by AZD1775 (formerly MK-1775) in combination with cisplatin resulted in significant apoptosis in multiple lung cancer cell lines.

WEE1 is an important cell cycle checkpoint kinase that negatively regulates the G2/M transition, This kinase interacted specifically with PAXIP1 (a.k.a PTIP) in the tBRCT PPIN screens. PAXIP1 has three tBRCT domains that coordinate foci formation at sites of DNA damage and is required for cells to progress to mitosis (FIG. 1). The apparent overlap of cell cycle regulation and molecular interaction between PAXIP1 and WEE1 prompted examination of the biological role of PAXIP1 in lung cancer response to WEE1 inhibitor treatment.

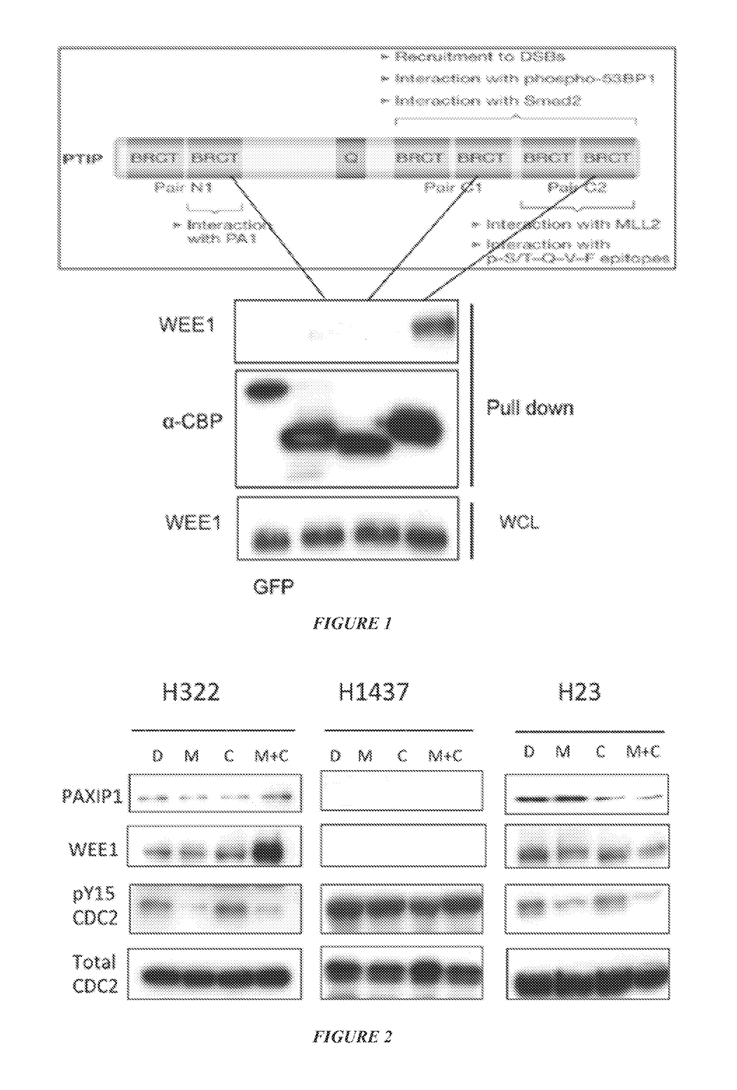

AZD1775 is an ATP mimetic that targets the kinase activity of WEE1 and is currently being evaluated in clinical trials for several tumor types. Phosphorylation of CDC2 (a.k.a. CDK1) by WEE1 can be used as a marker for WEE1 inhibition. In 15 lung cancer cell lines treated with AZD1775 as a single agent or in combination with cisplatin, it was Observed that presence of detectable (or elevated) PAXIP1 and WEE1 in combination correlate with inhibitor-dependent repression of CDC2 phosphorylation (FIG. 2).

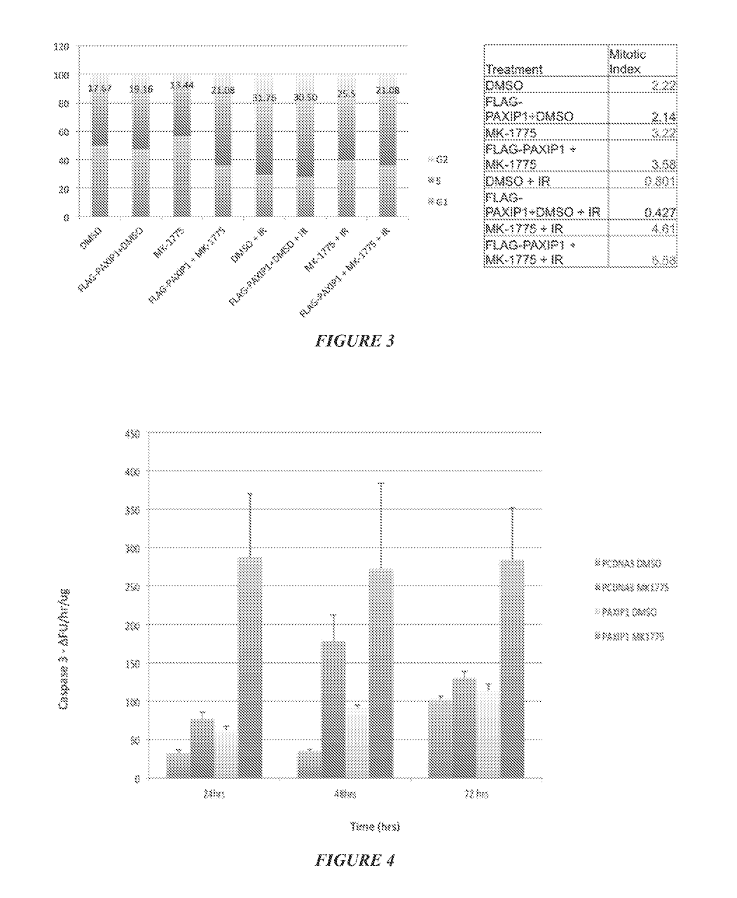

To analyze the effect of PAXIP1 on WEE1 inhibition, full length PAXIP1 was overexpressed in H322 cells, which led to an increased mitotic index upon WEE1 inhibition at the G2/NI checkpoint (FIG. 3). Overexpression of PAXIP1 also leads to sustained increase in caspase-3 mediated apoptosis when cells are treated with AZD 1775 over 72 hours compared to cells with low levels of PAXIP1 (FIG. 4). These results suggest that the levels of PAXIP1 could be used to identify cancers that would exhibit a favorable response to WEE1 inhibition.

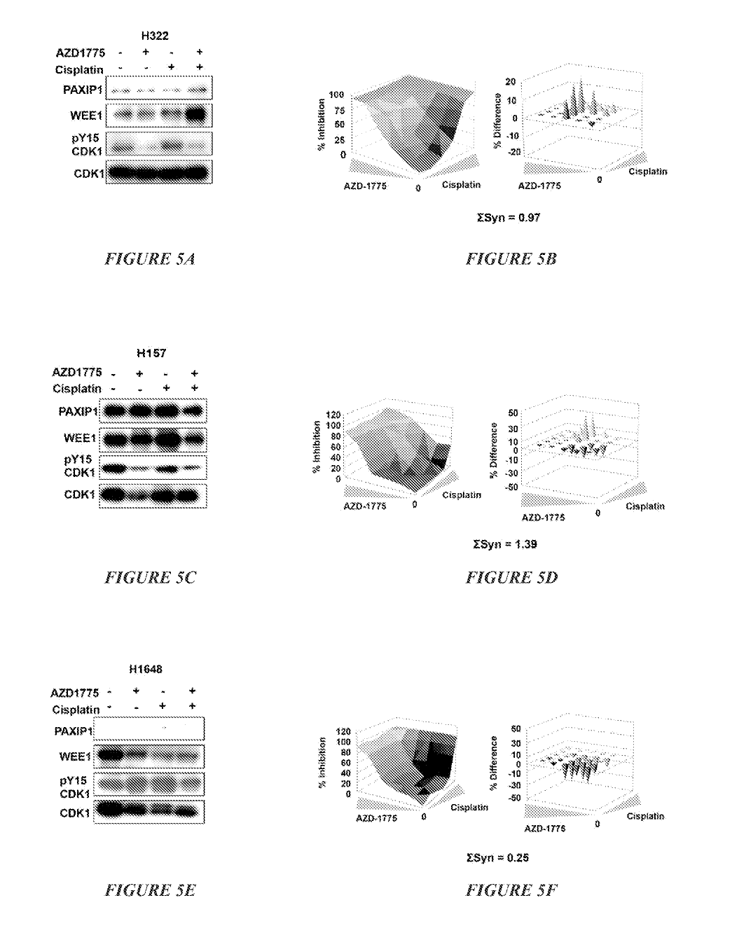

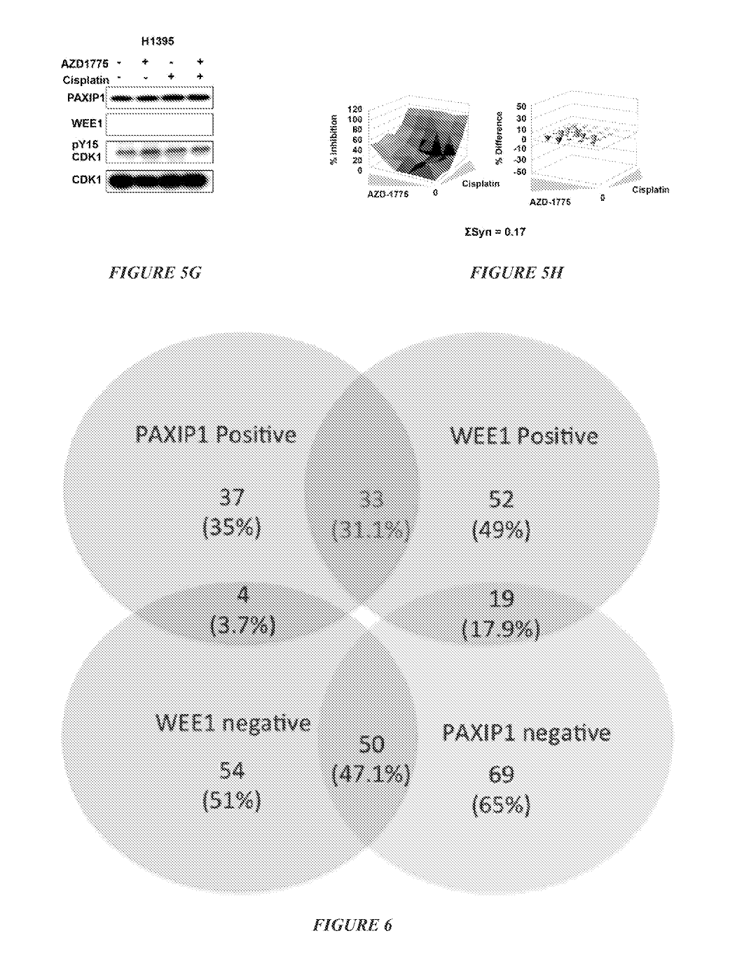

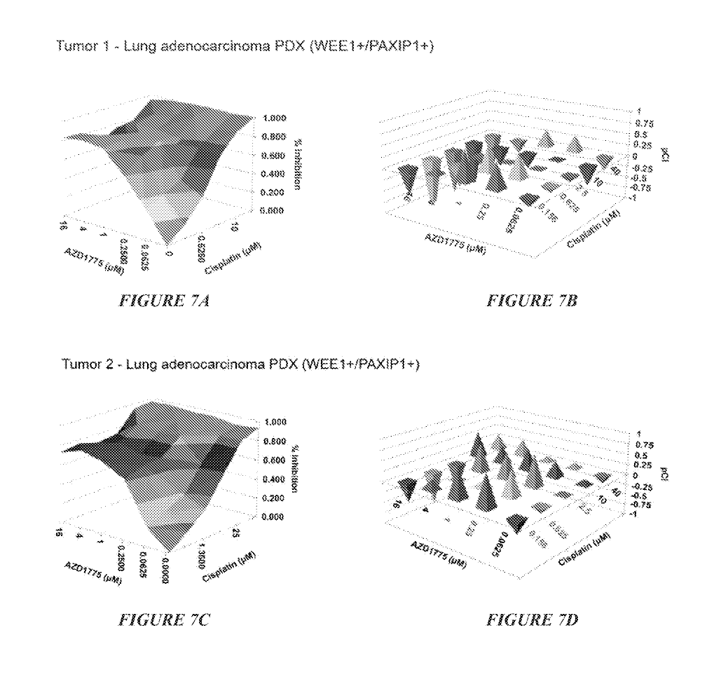

Additionally, PAXIP1 and WEE1 expression profiles were analyzed in lung tumor samples using tissue microarrays (FIG. 6). Approximately 30% of lung tumors are positive for both WEE1 and PAXIP1 expression. Combined with the in vitro data (FIG. 5), this group of tumors is expected to respond better to AZD1775 treatment.

PAXIP1 is also found altered (mutated/amplified/deleted) with a high frequency in ovarian cancers (˜12%). This implies that, in the future, PAXIP1 could also be used as a potential biomarker for WEE1 therapy in these cancers

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for selecting a cancer therapeutic for a patient, comprising
(a) assaying a tumor biopsy sample from the subject to detect PAXIP1 expression, and
(b) selecting a WEE1 inhibitor as the cancer therapeutic if PAXIP1 is detected in the tissue sample.

2. The method of claim 1, wherein the WEE1 inhibitor comprises AZD1775 (formerly MK-1775).

3. The method of claim 1, wherein the tumor biopsy is assayed by measuring PAXIP1 protein levels in the tumor biopsy sample.

4. The method of claim 1, wherein the cancer is a carcinoma.

5. The method of claim 1, wherein the cancer is a sarcoma.

6. The method of claim 1, further comprising treating the subject with a WEE1-sensitive antineoplastic agent along with the WEE1 inhibitor if PAXIP1 is detected in the tissue sample.

7. The method of claim 6, wherein the WEE1-sensitive antineoplastic agent comprises carboplatin or cisplatin.
